(19) United States
(12) Patent Application Publication
(10) Pub. No.: US 2012/0174320 A1
(43) Pub. Date: Jul. 12, 2012

(54) Body Shaped Comfort Pillow

(76) Inventors: Joni White Thomas, Convington, GA (US); Eulalee Gray, Covington, GA (US)

(21) Appl. No.: 13/291,944

(22) Filed: Nov. 8, 2011

Related U.S. Application Data
(60) Provisional application No. 61/432,053, filed on Jan. 12, 2011.

(51) Int. Cl. A47G 9/10; A47C 21/04; A61F 7/00

(52) U.S. Cl. 5/632; 5/630

(57) ABSTRACT

An anatomical body pillow designed to assist with the physical comfort of a practitioner lying on his or her side or back. The first embodiment comprises a torso portion with arm sections to support the user's neck, shoulders and head. The arm sections may be full-length or shorter sections, while the torso region includes quilt stitching to provide anatomical definition and a plurality of pockets. A second embodiment of the device includes a lower body pillow with a waist area and leg sections. The waist may be used as a pillow for a user's head while the leg portions act as arm supports. A faux skirt is attached to the waist region to provide an optional eye covering. Heat packs, cold packs, fragrance sachets and other devices may be inserted into pockets provided in both embodiments, supplying a user with physical and therapeutic comfort while resting in a prone position.

### CROSS REFERENCE TO RELATED APPLICATION

This application claims the benefit of U.S. Provisional Application No. 61/432,053 filed on Jan. 12, 2011, entitled “Emo-Comfort Pillow.”

### BACKGROUND OF THE INVENTION

1. Field of the Invention

The present invention relates to a sleeping body support pillow. More particularly the present invention is a body pillow having anatomical features specifically designed to provide support for the neck, back and shoulders while lying in a variety of positions.

Individuals tend to use pillows for a variety of purposes, most prominently to provide physical support while resting or in a prone position. The typical practitioner will use conventional, square pillows for sleeping and relaxing. While these pillows tend to be adequate for their intended purpose, they do not provide the ideal level of physical support required by some users. In particular, it is critical for individuals with injuries, physical conditions or chronic pain to have particularized support that provides improves sleeping posture and prevents certain body positions that can cause physical pain after prolonged periods. Many categories of individuals require specialized support or therapeutic administration to provide comfort, includes those with back pain, sore muscles, cramps and those with other specific bodily injuries.

The present invention discloses a support pillow designed to provide comfort and improved sleeping posture for all individuals. Of particular concern are individuals lying on their side and back. While on one's side, the shoulders of a user may tend to slump into the chest, wherein one shoulder is against a sleeping surface and another is otherwise unsupported. This collapsing of the chest results in a poor sleeping posture, which can lead to body aches and muscle cramps resulting from such an awkward position maintained for a prolonged period. While on one's back, it is necessary to support the head, neck and shoulders of a user. The present invention contemplates two embodiments of an anatomical body pillow that addresses these needs and alleviates potential problems with sleeping posture. By providing a stable cushioned support, the pillow maintains proper body and joint alignment, which reduces stress on muscles, ligaments and joints. Further, the present invention includes internal and surface pockets that allow the insertion of a variety of objections to assist in the therapeutic treatment of physical injuries, including thermal packs and aroma therapy devices.

A secondary need provided for the individual via the present invention is that of emotional security and comfort. The present invention provides a body pillow with anatomical features to contour to a user's body and complement their position while in a sleeping or resting position, either on their back or lying on their side. Individuals of all ages desire such security and while a pillow that provides physical comfort is important, it is necessary to have emotional comfort as well. Often the use of a body pillow will provide the user with emotional security and support. A pillow that is properly designed to address the physical support and emotional needs of its user is desirable.

The present invention provides an alternative to conventional pillows that is designed to fill both the physical support needs and the therapeutic needs of the practitioner, unlike currently available body pillow devices in the prior art. Further, the nature of the body pillow structure provides emotional support as well. The present invention is shaped in the likeness of the upper or lower portion of a human torso, which provides individuals with not only physical comfort and support, but emotional security as well. Further, to address any physical therapeutic needs, the device employs a variety of surface pockets to accommodate either hot or cold packs, heating pads or aroma therapy objects. The present invention fulfills a long felt need in the art by providing support for the arms, shoulders and neck in a manner which resembles a human body to improve sleeping posture and support for a user, wherein the body pillow may take the form of two anatomical structures resembling an upper or lower body portion of a human.

2. Description of the Prior Art

Several patents have been issued and applications published related to devices that act as pillows, however, these devices each have known drawbacks or fail to entirely address the issues of the present invention. Several devices have been designed to serve as therapeutic pillows or to provide comfort to the user when oriented in certain positions or postures. Other devices provide hot or cold comfort therapy. However, the prior art devices do not address both the issues of physical and emotional comfort, along with the anatomical features disclosed by the present invention.

Several previous devices have been designed to serve physical and therapeutic functions, such as comforting specific muscles or strategically placing hot or cold packs to provide relief. One such device, Russell U.S. Patent Publication No. 2008/0066233 is directed to a neck therapy cushion or pillow that can include a t-shaped porous enclosure formed from fabric and a plurality of bead-like members within the enclosure. The porous fabric can be made from cotton or a cotton blend. The plurality of bead-like members can be a plurality seeds such as flax or sesame seeds, synthetic foam beads, or other materials can be placed within the enclosure. Herbs such as lavender, peppermint or orange peel can also be placed or retained within the enclosure. A portion of the t-shaped porous enclosure can fit under a person's neck while a straight portion can lie under the person's spine while the person is lying down. Additionally, the cushion may be heated or cooled to provide the appropriate healing effect.

Similarly, Simmons U.S. Pat. No. 4,783,866 is directed to a therapy pillow having a cold pack specially constructed and contoured to provide intimate contact with the occipital region of the head. The pillow is constructed and contoured to fit partially around the neck and tapered such that the surface portion of the pillow will apply force to the occipital region of the head. A pocket provided in the pillow receives a specially contoured container filled with a temperature retaining material providing a cold pack that will relieve migraine or muscular contraction headaches when applied to the head. The specially constructed pocket is lined with a moisture resistant material and is easily and quickly opened and closed for receiving or removing the cold pack.

Further, Pistay U.S. Pat. No. 5,344,437 is directed to a therapeutic massaging pillow. The pillow is provided in a U-shaped form having extensions that fit around the neck and a thicker upper portion that is in intimate contact with the posterior portion of the head of a person using the pillow. The pillow provides a pocket on one or both sides for receiving a container that is filled with a material that can be heated or cooled. Additionally, a cavity at the thickest upper portion of the pillow is provided for installing an electrically operated vibrator. The vibrator is embedded in a foam housing and has an easily accessible switch. The pillow allows application of hot or cold temperatures to the posterior of the head through the fluid filled container inserted in the pocket or separately applying the massaging effect of the vibrator.

The Russell, Simmons and Pistay devices are all described as physically therapeutic pillows that provide relief for physical discomfort. Such devices are useful with physical soreness, allowing the application of treatment to specific areas, but fail to address a need for physical support. Such devices are not designed to support the back, neck and shoulders in a variety of positions. The present invention is configured to provide both therapeutic comfort as well as physical support to the user while lying on his or her side or back.

Other prior devices address the issue of sleeping comfort, by providing securable or configurable body pillows. Kelly U.S. Pat. No. 6,052,848 is directed to a body support pillow stuffed with resilient deformable filler and having a U-shaped central section and legs. The pillow has a center opening equal to the body width of a user. In a first embodiment, the pillow's total length is substantially equal to the height of a user, and the legs are selectively foldable over one another to create a double thickness for additional support under the user's knees, with a detachable body-encircling strap securing the pillow about the user.

Similarly, Leach U.S. Pat. No. 6,499,164 is directed to a body pillow having an upper end which is essentially in the shape of a horseshoe and a lower end which is essentially in the shape of a “J”, a straight portion connecting the horseshoe-shaped top with the “J”-shaped bottom.

Further, Leach U.S. Pat. No. 6,751,817 is directed to a contoured body pillow adapted to permit unlimited positioning options to facilitate relief from persistent back and neck pain, respiratory and digestive disorders, and to provide a sensation of closeness, comfort and security by conforming to the body of a user. The device permits the user to sleep comfortably through the night and has a substantially candy cane shape. The pillow comprises a first terminal end having a semi-bell shape connecting to a U-shaped portion, a downwardly extending leg connecting to the U-shaped portion, the leg expanding at its lowermost portion to form a bell shape, the bell shape terminating at a second terminal end in a beveled edge.

The Kelly and Leach devices each describe body pillows designed to facilitate relief from back, neck and other bodily pains. While these devices serve to assist the user is achieving physical comfort, they are aimed at treating injuries or conditions. These inventions do not address a need for physical comfort and support. Further, these prior inventions do not demonstrate any therapeutic means. Conversely, the present invention provides a supportive pillow companion with pockets for therapeutic applications.

It is therefore submitted that the present invention is substantially divergent in design and purpose from the prior art, consequently it is clear that there is a need in the art for improvement and innovation to existing body pillow devices. In this regard, the instant invention substantially fulfills these needs.

### SUMMARY OF THE INVENTION

In view of the foregoing disadvantages inherent in the known types of body pillows now present in the prior art, the present invention provides a new physically supportive and comforting body pillow wherein the same can be utilized for providing convenience for the user when positioning themselves in a manner requiring support for their arms, shoulders or neck.

It is therefore an object of the present invention to provide a new and improved physically supportive and comforting body pillow device that has all of the advantages of the prior art and none of the disadvantages. It is a particular object of the present invention to provide support to the neck, shoulders and back while the user is lying down, either on their side or on their back.

Another object of the present invention to provide a body pillow that is supportive of a plurality user positions, wherein the two embodiments of the body pillow provide support for a user either lying on his or her back or side.

Another object of the present invention is to provide a body pillow containing pockets and sleeves to allow for the insertion of heating and cooling objects to further the emotional, therapeutic and physical comfort of a user. Such objects may include ice packs or heating pads to alleviate muscle or joint pain. Alternatively, such objects could include scented or fragrant objects to soothe emotional and physical stress.

Yet another object of the present invention is to provide physical comfort and support when the practitioner is in a sitting or lying position. A further related object of the invention is to provide comfort and prevent aches and soreness while sleeping or resting.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 is a perspective view of the upper torso embodiment of the present invention, wherein an anatomical torso pillow with arms is provided.

FIG. 2 is a perspective view of the upper torso embodiment of the present invention in use with a user lying on her side.

FIG. 3 is a perspective view of the lower body embodiment of the present invention.

FIG. 4 is a perspective view of the lower body embodiment of the present invention in use with a user lying on his back.

FIG. 5 is a perspective view of the lower body embodiment of the present invention wherein a back support portion is provided between the first and second legs of the pillow.

### DETAILED DESCRIPTION OF THE INVENTION

Reference is made herein to the attached drawings. Like reference numerals are used throughout the drawings to depict like or similar elements of the anatomical body pillows. For the purposes of presenting a brief and clear description of the present invention, the preferred embodiment will be discussed as used for providing emotional and physical comfort through the use of a body pillow device. The figures are intended for representative purposes only and should not be considered to be limiting in any respect.

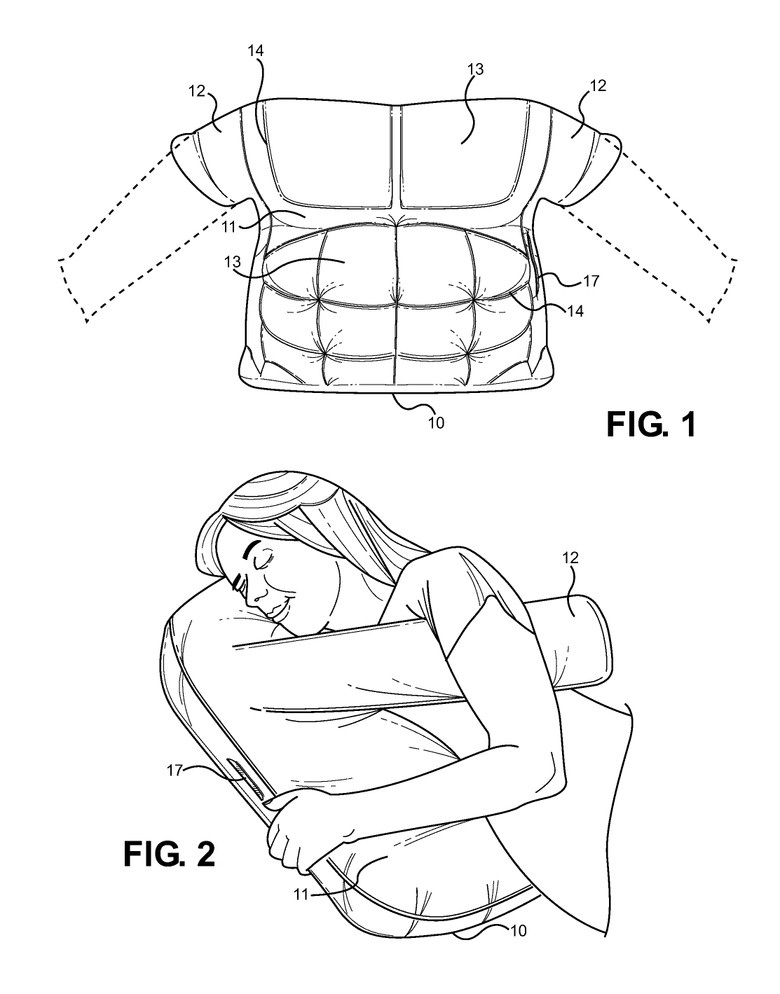

Referring now to FIG. 1, there is shown a perspective view of a short-armed torso embodiment of the present invention. The pillow structure 10 is defined by a human torso-shaped body portion 11 with arm projections 12 of desired length. Quilted stitching 14 along the body portion 11 define a variety of anatomical features, as well as a plurality of pocket locations 13 into which therapeutic or thermal packs may be placed. These pockets 13 are placed strategically about the torso region for contact with injured body parts on the practitioner's body or for providing warmth to simulate the presence of another. The arm projections 12 may be provided in a plurality of lengths, including a short armed, long armed or no-armed torso embodiment. The alternative long armed embodiment is demonstrated with dashes outlining extended arm portions 12. Along the side of the body portion 11 is an enlarged pocket 17 for placement of larger thermal or aromatherapy packs within the structure of the device.

Referring now to FIG. 2, there is shown a perspective view of a long-armed torso embodiment of the present invention in use, wherein a large side pocket 17 is provided for thermal or aromatherapy device insertion. In this embodiment of the present invention, the torso body portion 11 includes two long-armed projections 12. This embodiment allows the arms 12 to be used as further body supports and comfort. In FIG. 2, the position of the user with respect to the torso body pillow is shown in its working position. The pillow device is adapted to allow a user to rest on his or her side, with a first shoulder bearing against a support surface and a second shoulder supported by the body of the torso pillow. In this way, the second shoulder is prohibited from “rolling” inward, wherein it would roll forward and collapse the chest of the user, placing his or her body in a position of poor posture. The torso body pillow supports the second shoulder in a static position to prevent this motion, and thus improving the user's support while sleeping and preventing injury while lying on one's side. This prevents aches or cramps after prolonged positioning in an otherwise unsupported side position.

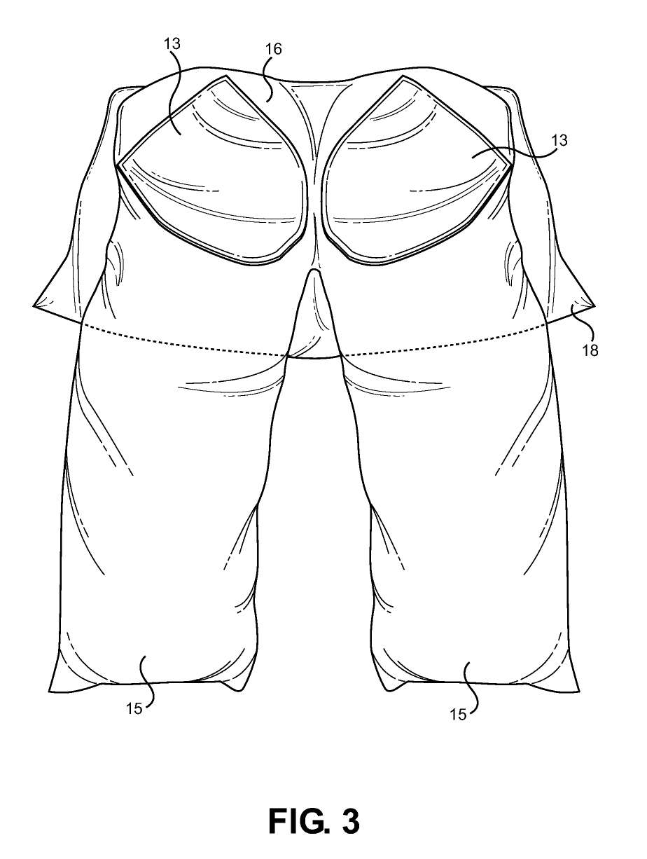

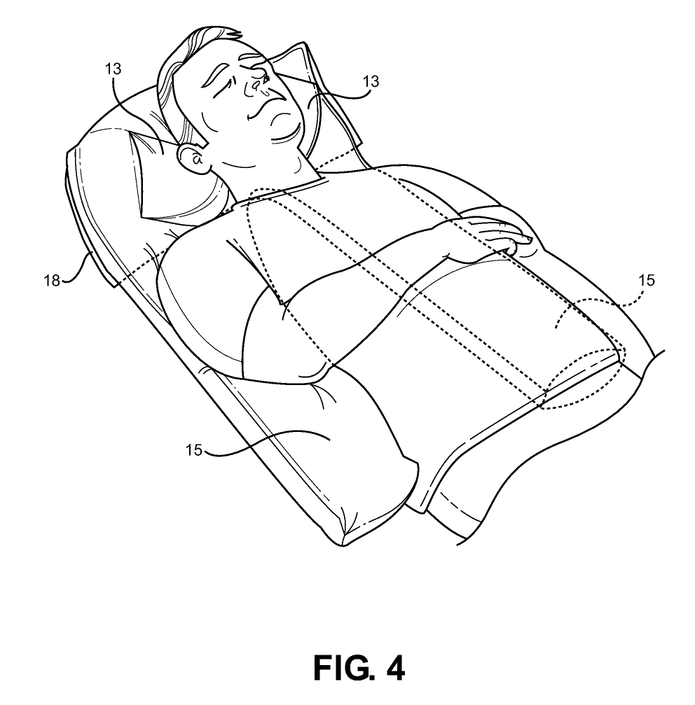

Referring now to FIG. 3, there is shown a perspective view of the lower body embodiment of the present invention, wherein an anatomical structure comprising two legs 15 and a waist region 16 are provided. The pillow structure is adapted to provide a split-body pillow that supports a user lying on his or her back. When lying on his or her back, the practitioner's head is supported on the waist region 16, while the leg structures 15 support the user's arms and shoulders. The split between the legs is used to accommodate the body of the user, with the legs conforming to and supporting both sides of a user's upper body. The device provides support for the arms, shoulders and neck, and prevents painful and may be utilized to support the head of the user in an elevated position. A further aspect of this embodiment is the inclusion of a simulated skirt 18, which is connected to the waist structure 16 at a first end, while its second end may be draped over a user's eyes, providing an eye cover to increase darkness to assist with sleep. The skirt 18 attaches to a first side of the waist region 16, while the user's head is adapted to be placed on the opposite, second side of the waist region. The second side incorporates a plurality of pockets 13 for heating pads, aromatherapy sachets or similar devices to facilitate pain or symptom relief or improved sleep of the user. The skirt 18 is folded over the end of the waist region and over a user's eyes, if so desired by the user.

Referring now to FIG. 4, there is shown a perspective view of the lower body embodiment of the present invention in use, wherein a user is lying on his back with the legs 15 of the device supporting his arms and shoulders, while the waist portion is support his head. In this position, the user's head is supported in proximity to pockets 13 located on the waist region, which allow sachets or thermal packs to be inserted as desired. In this view, the eye-covering skirt 18 is shown in a stowed position, wherein it is folded around the backside of the waist and is not in use. If desired, this skirt 18 may be folded over the user's head to cover his eyes while sleeping, providing increased darkness that certain users may desire.

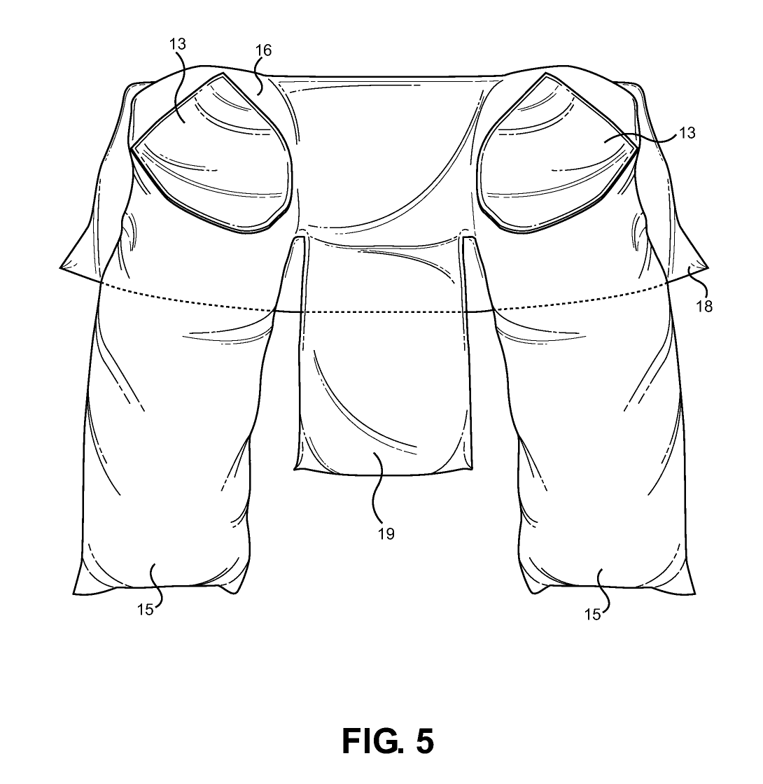

Referring now to FIG. 5, there is shown an alternate form of the lower body embodiment of the present invention, wherein a back support pillow 19 is provided between the two legs 15 of the device. The back support pillow 19 provides a padded region that supports the user's back while sleeping face-up. This extra pillow provides extra padding between the user and a support surface, while the user's arms are supported by the legs 15 and the user's neck and head are supported by the waist region 16. The back support 19 lies between the pair of legs and is attached to the waist region 16. If undesired, this portion may be folded under the waist region 16 to further lift the user's head and remove padding beneath the user's back.

The present invention is designed to be an improvement upon existing body pillows, and one that improves the posture and delivery of therapy to a user in lying on his or her side or back. One embodiment of the device is designed to function as an upper body pillow while the user is positioned on his or her side. This embodiment comprises a body pillow structure in the shape of a human torso filled with cushioning material and having anatomical features stitched into its structure to resemble a human torso. The shape of the body pillow is T-shaped to simulate with a body portion and arm projections. Built into the lining of the body pillow structure 10 are a plurality of pockets designed to hold therapeutic devices such as hot and cold packs, fragrance sachets and chemical heating pads. The pockets may be situated within the stitched regions of the torso, while a larger pocket is provided within the structure of the pillow. Access to this larger pocket is provided along a side of the device, wherein a larger therapy pad or article may be placed therein. Locations for insertion of therapy device may be chosen such that the pockets lie against potentially sore or critical portions of the user's body. The pockets are adapted to include a fastening means such that the articles placed therein are retained in position. Such a fastening means preferably includes hook and loop fastening, buttons or snap closure. The pockets are sewn into a liner of the pillow structure, each pocket having its own cavity for securement of an article. The larger opening of the body pillow structure is located on the side of the device and includes a large, internally lined pocket. The larger internal pocket runs nearly the length and width of the torso body portion of the device, allowing insertion of a large object, such as a larger heating pad or further cushioning material. Insertion of a large thermal pad effectively allows the device to simulate a companion or to provide therapeutic hot or cold temperatures to an affected area. The body pillow structure includes quilt-style stitching that outlines a chest, abdomen, and arms, and further defines the pockets of the device. Both the front and back of the pillow may have an outlines of anatomical features. The shape of the torso embodiment of the present invention is most suitable for replacing a traditional body pillow, providing support to the shoulders, arms and back while lying on one's side. In this position, the user's shoulders and chest are supported and prevented from collapsing into a position of poor posture. This is of particular concern for those with core body injuries or lack of strength. This embodiment may be provided with short, long or nonexistent arm projections.

A second embodiment of the present invention functions as an upper body back and neck support pillow, comprising body pillow structure having anatomical features resembling a pair of legs and a waist region. This pillow is adapted to be placed behind a user lying on his or her back. The user's head is supported along the waist region, while the legs of the device function as shoulder and arm supports, allowing for full upper body support of a user. The legs spread apart on either side of the user's head, supporting his or her shoulders and arms while lying thereupon. An optional back support pillow may further be provided between the pair of legs of the pillow, while a plurality of pockets is provided along the user-side of the device, adapted to secure thermal pads or therapeutic device against the user's neck or back. The pockets may include a fastening means to hold in and to secure objects, such a means may include hook and loop fastening, button or snap closure. The body pillow structure includes quilt-style stitching that outlines the muscles and features of the waist region and legs of a person, and further defines the pockets of the device. Both the front and back of the pillow may have an outlines of anatomical features.

The surface of the pillow may be cotton, silk, or a quilt material, while the inside may be filled with polyester fiberfill, memory foam or any other suitable type of material known to one skilled in the art of pillows. Alternative embodiments of the present invention may include variations in torso pillow arm length, or no arms. Variations may further include a pillow structure having a multitude of smaller surface insertion pockets, a larger internal pocket or both. The pillow may similarly may constructed in a variety of sizes, to allow individuals to choose the appropriate substitute for their support pillow or the ideal body structure sized to accommodate the user's body type.

In use, the lower body embodiment of the body pillow structure operates in a slightly different manner. This embodiment is designed for the practitioner who prefers to sleep on their back. The waist portion 16 of the embodiment is used a head and neck pillow and support, while the leg portions 15 act as arm supports. As with the previous embodiment, pockets 13 may be filled with a variety of therapeutic devices. Additionally, another aspect of this embodiment is the inclusion of a faux simulated skirt 18, which may be extended and pulled over the practitioner's eyes to provide additional darkness for comfortable sleep. The embodiment also provides emotional comfort through its physical support and therapeutic enhancements.

To this point, the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim

1) An anatomically shaped body pillow device, comprising:
a human torso shaped body pillow structure with a first and second, an interior volume and a shoulder region;
said interior volume filled with cushion material,
two arm projections attached at said shoulder portion;
quilted stitching on said torso shaped body pillow structure and said arm portions so as to create anatomical features of a human;
a plurality of pockets along said body pillow structure into which a therapeutic device may be inserted.

2) The device of claim 1, wherein said torso shaped body pillow structure further comprises a large internal compartment, said compartment being accessible via a large pocket on said pillow structure side.

3) The device of claim 1, wherein said arm portions are full arm length, providing physical support for the arms, neck and shoulders.

4) The device of claim 1, wherein said arm portions are less than full arm length.

5) The device of claim 1, wherein said plurality of pockets employ an individual internal liner within the body pillow structure.

6) The device of claim 1, wherein said cushion material comprises memory foam or polyester fiberfill.

7) The device of claim 1, wherein said therapeutic device comprises a thermal pad device.

8) The device of claim 1, wherein said therapeutic device comprises a fragrance sachet.

9) An anatomically shaped body pillow device, comprising:
a human lower body shaped pillow structure having a first and second side, an interior volume, a waist region and two leg portions;
said interior volume filled with cushion material;
quilted stitching on said body pillow structure and said leg portions so as to create anatomical features of a human;
a plurality of pockets along said pillow structure first side into which to place a therapeutic device.

10) The device of claim 9, further comprising a back support region between said leg portions and extending from said waist region.

11) The device of claim 9, further comprising a skirt attached to said pillow structure second side, which may be extended over a practitioner's eyes to provide additional darkness during sleep.

12) The device of claim 9, wherein said cushion material comprises memory foam or polyester fiberfill.

13) The device of claim 9, wherein said therapeutic device comprises a thermal pad device.

14) The device of claim 9, wherein said therapeutic device comprises a fragrance sachet.
